SEQUENCE LISTING (8 sequences)

SEQ ID NO: 1 - protein, 13 aa, Influenza A virus
PKYVKQNTLKLAT

SEQ ID NO: 2 - protein, 11 aa, Influenza A virus
GLFGAIAGFIE

SEQ ID NO: 3 - protein, 12 aa, Artificial Sequence
  Synthetic
GLLGAIAGPIEF

SEQ ID NO: 4 - protein, 12 aa, Artificial Sequence
  Synthetic
GLMGAIAGPIEF

SEQ ID NO: 5 - protein, 14 aa, Artificial Sequence
  Synthetic
GLLGAIAGPIEGGW

SEQ ID NO: 6 - protein, 14 aa, Artificial Sequence
  Synthetic
GLHGAIAGLIENGW

SEQ ID NO: 7 - protein, 11 aa, Artificial Sequence
  Synthetic
  misc_feature (3)..(3): Xaa can be any naturally occurring amino acid
  misc_feature (9)..(9): Xaa can be any naturally occurring amino acid
GLXGAIAGXIE

SEQ ID NO: 8 - protein, 12 aa, Artificial Sequence
  Synthetic
LLVEAAPLDDTT

(19) United States
(12) Patent Application Publication
(10) Pub. No.: US 2021/0052719 A1
(43) Pub. Date: Feb. 25, 2021

(54) A UNIVERSAL VACCINE AGAINST INFLUENZA

(71) Applicant: Development Center for Biotechnology, Taipei City (TW)

(72) Inventors: Jia-Ming CHANG, Taipei City (TW); Wan-Jyun WEI, Taipei City (TW)

(73) Assignee: Development Center for Biotechnology, Taipei City (TW)

(21) Appl. No.: 16/959,117

(22) PCT Filed: Dec. 29, 2017
(86) PCT No.: PCT/US2017/069132

§ 371 Date: Aug. 23, 2020

(51) Int. Cl. A61K 39/145; C07K 14/005

(52) U.S. Cl. CPC A61K 39/145; C07K 14/005; A61K 2039/575; C12N 2760/16134; C12N 2760/16122

(57) ABSTRACT

An antigenic short peptide includes 11 to 15 amino-acid residues and has an ability to induce antibody against influenza virus. The sequence of the antigenic peptide is selected from hemagglutinin (HA). The antigenic peptide includes the sequence of JJ (SEQ ID NO:2), JJ-1 (SEQ ID NO:3), JJ-2 (SEQ ID NO:4), JJ-3 (SEQ ID NO:5) or JJ-4 SEQ ID NO:6). A method for inducing a broad-spectrum immunity against influenza viruses includes administering a vaccine to a subject, wherein the vaccine comprises one of the above antigenic peptide.

### FIELD OF THE INVENTION

The present invention relates to vaccines against influenza, particularly to peptide-based broad-spectrum vaccines.

### BACKGROUND OF THE INVENTION

Influenza continues to pose a threat to human health mainly due to the imperfect protection of vaccines and the gradual spread of resistance to anti-viral drugs. Among them, the flu viruses from farm animals may cause fatalities in humans when these viruses gain the abilities to infect human. Such infection not only causes economic loss to the source of food, but also threatens human health. Due to the diversity of influenza viruses, it is impossible to protect against a majority of influenza viruses.

Influenza viruses use hemagglutinin (HA) to bind the target cells. The name “hemagglutinin” comes from the protein's ability to cause red blood cells (erythrocytes) to clump together (agglutinate) in vitro. HA comprises two disulfide-linked subunits, HA1 (head) and HA2 (stem). The HA1 “head” subunit is responsible for binding of viruses to the target cells by interactions with sialic acid on the target cells. After binding, the virus is endocytosed into the cells. The acidic environment of endosomes triggers conformational changes of the HA2 “stem” subunit, leading to fusion of the viral membrane and endosomal membrane. As a result, the viral genome is released into the cytoplasm, allowing the infection to progress.

Because HA is essential for virus to infect cells, HA is a target for intervention. For example, neutralizing antibodies can prevent virus binding to cells, thereby preventing virus infection. However, due to strain variations of influenza viruses, there are many subtypes of hemagglutinins and vaccines typically are only effective against homologous virus strains. Development of broad-spectrum vaccines against influenza viruses is highly desirable.

Even though there have been efforts to generate broad-spectrum vaccines, there is still a need for a safer and more effective vaccine against various types of influenza virus.

### SUMMARY OF THE INVENTION

Embodiments of the invention relate to universal vaccines against influenza viruses. Vaccines of the invention are peptide-based and are effective against a wide range of influenza virus subtypes.

One aspect of the invention relates to antigenic short peptides. An antigenic short peptide in accordance with one embodiment of the invention includes 11 to 15 amino-acid residues and has an ability to induce antibody against influenza virus. The sequence of the antigenic peptide is selected from hemagglutinin (HA). The antigenic peptide includes the sequence of JJ (SEQ ID NO:2), JJ-1 (SEQ ID NO:3), JJ-2 (SEQ ID NO:4), JJ-3 (SEQ ID NO:5), and JJ-4 (SEQ ID NO:6).

One aspect of the invention relates to methods for inducing a broad-spectrum immunity against influenza viruses. A method in accordance with one embodiment of the invention includes administering a vaccine to a subject, wherein the vaccine comprises one of the above antigenic peptide.

Other aspects of the invention will become apparent with the following description, the drawings, and the accompanied claims.

### BRIEF DESCRIPTION OF THE DRAWINGS

FIG. 1 shows structures of hemagglutinin illustrating location of the antigenic peptides.

FIG. 2A shows induction of antibody (specific anti-JJ IgG) by peptide JJ of the invention. FIG. 2B shows induction of antibody (specific anti-JJ IgG) by peptide JJ-1 of the invention. FIG. 2C shows induction of antibody (specific anti-JJ3 IgG) by peptide JJ-3 of the invention.

FIG. 3 shows antibody titers against various subtypes of hemagglutinins.

FIG. 4 shows results of hemagglutination inhibition by antibodies of the invention.

### DETAILED DESCRIPTION

Embodiments of the invention relate to universal vaccines against influenza viruses. Embodiments of the invention are peptide vaccines derived based on viral hemagglutinins (HA), wherein the peptides may be resistant to protease digestions such that these vaccines may be used as oral vaccines.

In accordance with embodiments of the invention, the peptide sequences are selected from the sequences of viral hemagglutinins. The selection is based on computer modeling of potential peptide sequences binding to MHC class II. The computer modeling also analyzes potential glycosylation sites. The selected peptide sequences preferably are away from the glycosylation sites on HA.

A number of computational approaches are available for predicting peptide-MHC binding. See for example, Buus (1999) and Robinson et al. (2003).

After computer modeling, the peptides may be synthesized and tested for their binding to the MHC molecule. The binding assays, for example, may be performed with ELISA.

Major histocompatibility complex class II (MHC-II) are transmembrane heterodimeric proteins on the surface of antigen presenting cells (APCs). They are essential for immune response to foreign antigens, by binding and presenting antigenic peptides to CD4⁺ T lymphocytes.

For example, after infection by influenza viruses, some viral proteins (e.g., hemagglutinin) may be processed by antigen-presenting cells (APCs). After processing by APCs, the antigenic peptides bind MHC class II molecules. The peptide-MHC complexes are then presented on the surfaces of the APCs, and the complexes interact with CD4⁺ T cells to trigger the immune responses. A particular fragment of HA, PKYVKQNTLKLAT (SEQ ID NO: 1), corresponding to the residues 307-319 of hemagglutinin (H3 subtype), from influenza A virus has been shown to bind with high affinities with certain subtypes of MHC class II molecules (see TABLE 1).

TABLE 1
affinity of MHC class II for antigenic peptide.

| Sequence | HLA-DR | Affinity (nM) |
|---|---|---|
| Positive sequence | | |
| for MHC class II | | |
| PKYVKQNTLKLAT | HLA-DRB1*101, *401, *701, *901, *1101 | 7.9~192.1 |
| | HLA-DRB1*1302 | |
| | HLA-DRB4*101 | 491.7 |
| | HLA-DRB5*101 | 36.7 |
| | HLA-DPA1*0201-DPB1*0101 | 490.4 |
| | HLA-DPA1*0103-(HLA-DPB1*0301/ | 0.1706 |
| | DPB1*0401) | |
| | HLA-DPA1*0301-DPB1*0402 | 166.7 |
| Natural sequence | | |
| GLFGAIAGFIE | HLA-DRB1*101, *701*901 | 169.5~404.6 |
| | HLA-DRB5*101 | 535.7 |
| | HLA-DPA1*0103-(HLA-DPB1*0301/ | 0.2122 |
| | DPB1*0401) | |
| | HLA-DQA1*0501-DQB1*0301 | 13.4 |
| Modified sequence | | |
| JJ-1 | HLA-DRB1*101, *701, *901, *1302 | 10.6~82.0 |
| | HLA-DRB5*101 | 382.2 |
| | HLA-DPA1*0103-(HLA-DPB1*0301/ | 0.1211 |
| | DPB1*0401) | |
| | HLA-DQA1*0501-DQB1*0301 | 8.6 |
| JJ-2 | HLA-DRB1*101, *701, *1302 | 13.3~96.0 |
| | HLA-DRB5*101 | 311.0 |
| | HLA-DPA1*0103-(HLA-DPB1*0301/ | 0.1153 |
| | DPB1*0401) | |
| | HLA-DQA1*0501-DQB1*0301 | 7.9 |
| JJ-3 | HLA-DRB1*101, *701, *901, *1302, | 11.6~210.5 |
| | HLA-DRB5*101 | 121.0 |
| | HLA-DPA1*0103-(HLA-DPB1*0301/ | 0.0913 |
| | DPB1*0401) | |
| | HLA-DQA1*0501-DQB1*0301 | 6.9 |
| JJ-4 | HLA-DRB1*101, *701, *901 | 12.7~238.1 |
| | HLA-DRB5*101 | 204.0 |
| | HLA-DPA1*0103-(HLA-DPB1*0301/ | 0.1171 |
| | DPB1*0401) | |
| | HLA-DQA1*0501-DQB1*0301 | 7.6 |

The 3D simulation structures of peptide-complexed WWII molecules revealed that peptide-MHC binding relies on interactions between pockets lining the WIC class II groove and side chains of the peptide, and a series of hydrogen bonds between nonpolymorphic MHCII side chains and the peptide backbone (Nelson et al., “Structural Principles of MHC class II Antigen Presentation,” Rev. Immunogenet., 1999, 1(1): 47-59).

By modeling peptide bindings to the MHC class II molecules, we have identified a new peptide sequence (“peptide JJ”; GLFGAIAGFIE, SEQ ID NO: 2) from HA that can also bind MHC class II molecules with high affinities. Several modeling approaches are available for analyzing peptide-MHC class II bindings. See for example, IEDB “MHC-II Binding Predictions,” http://tools.immuneepitope.org/mhcii/.

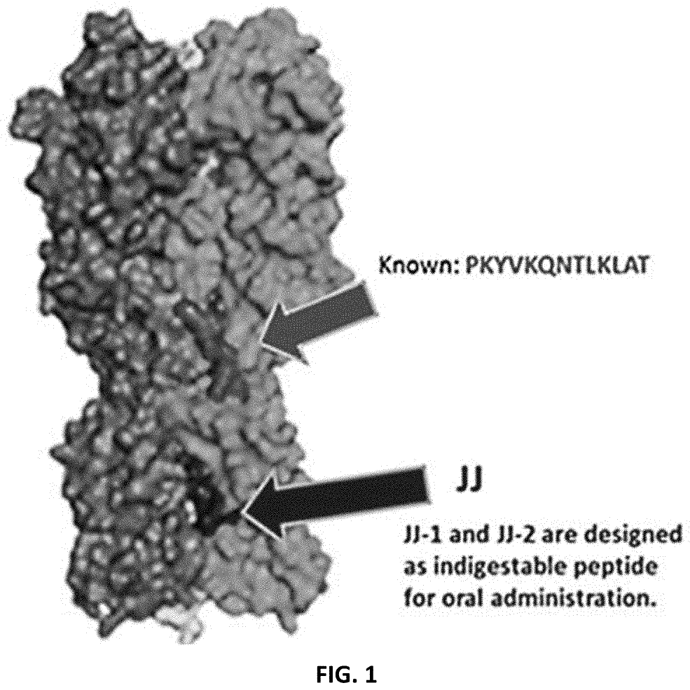

As shown in FIG. 1, this JJ peptide is located in a different region on the stem potion of the HA molecules, as compare with that of PKYVKQNTLKLAT (SEQ ID NO: 1). Furthermore, this JJ peptide sequence (GLFGAIAGFIE, SEQ ID NO: 2) represents a consensus sequence for various subtypes of hemagglutinins. Therefore, this sequence represents a promising epitope for broad-spectrum antibodies that can bind various subtypes of hemagglutinins.

We have also analyzed the potential glycosylation sites on HA because that might interfere with antibody bindings. The region of JJ peptide does not seem to have such potential glycosylation interference. Therefore, antibodies against this epitope region should not have this issue. This is corroborated by observations that antibodies generated with JJ peptide can bind to various subtypes of hemagglutinins, as described in a later section.

Based on the modeling, the JJ peptide is expected to bind tightly with MHC class II. This is corroborated by the fact that these peptides can induce antibody formations when used to immunize animals.

In accordance with embodiments of the invention, the peptides may be used as vaccines against influenza infection. The peptide vaccines may be administered via any suitable routes, such as by injection or per oral. In order to make effective oral vaccines, the peptide antigens should be able to withstand the environments in the digestive system. Therefore, based on JJ peptide, protease resistant peptides are designed. The protease resistance may be achieved by any method known in the art, for example by removing protease cleavage consensus/recognition sequences or by replacing natural amino acid residues with non-natural amino acid residues (e.g., chemically modified amino acids or D-amino acids).

As an example, one may be able to substitute amino acid residues in the JJ peptide to remove protease cleavage recognition sequence. Based on structural analysis of peptide-MHC-II complexes, the major binding interactions between the peptide and the MHC class II groove involve P1, P4, P6, and P9 sites, wherein the P1 site is located at the N-terminal side. Because P2, P3, P5, P7, and P8 sites are less important for MHC class II bindings, the amino acid residues of JJ peptide corresponding to these sites may be modified without significantly compromising the binding interactions with MHC class II. Therefore, one may be able to substitute residues at these sites to remove known protease cleavage consensus/recognition sequence.

In accordance with embodiments of the invention, protease-resistant peptides may be designed based on the JJ peptide (GLFGAIAGFIE) (SEQ ID NO: 2). Inventors of the invention have found that substitution of the phenylalanine (F) residues in JJ peptide can substantially eliminate its protease susceptibility. Three protease-resistant JJ peptide analogs have been thus obtained: JJ-1 (GLLGAIAGPIEF) (SEQ ID NO: 3), JJ-2 (GLMGAIAGPIEF) (SEQ ID NO: 4)], JJ-3 (GLLGAIAGPIEGGW) (SEQ ID NO: 5), and JJ-4 (GLHGAIAGLIENGW) (SEQ ID NO: 6). These peptides are investigated for their abilities to induce antibodies (i.e., to function as vaccines). Indeed, these peptides are found to bind MHC class II molecules with high affinities. (see TABLE 1).

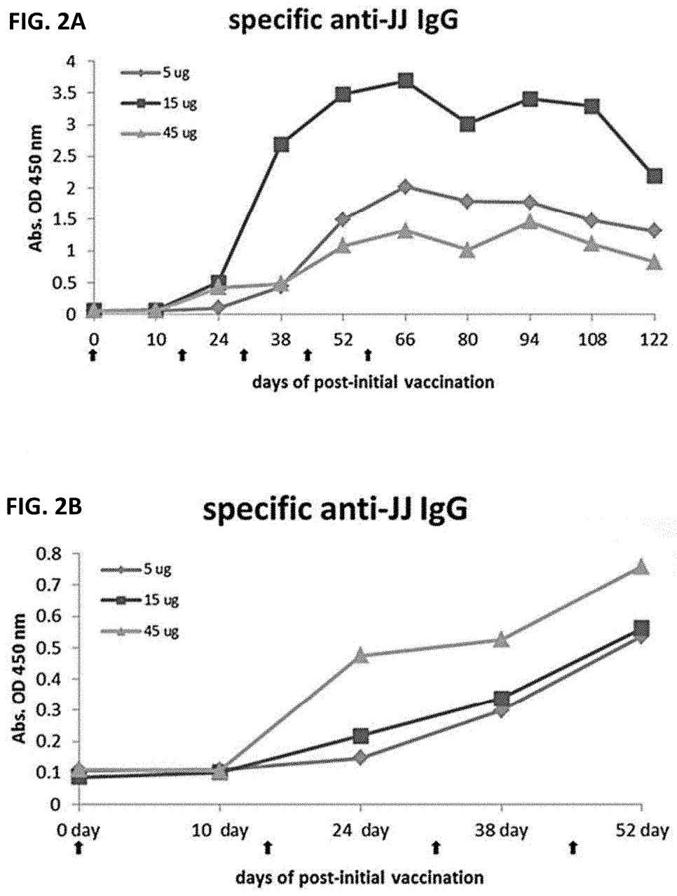

In addition, these peptides were found to induce antibodies against HA with high efficiency when these peptides are used to immunize mice. As shown in FIG. 2A, peptide JJ induced antibody (IgG) production in 3 different dose conditions (5 μg, 15 μg, or 45 μg dose for each group). Similarly, FIG. 2B shows that peptide JJ-1 efficiently induce IgG production in 3 different dose conditions (5 μg, 15 μg, or 45 μg dose for each group). These results indicate that peptide vaccines of the invention indeed are capable of inducing the production of antibodies. FIG. 2C shows induction of antibody production by peptide JJ-3 at three different doses (5 μg, 15 μg, or 45 μg). ELISA, with peptide JJ-3 coated on plates, clearly showed that at higher doses (15 μg and 45 μg), peptide JJ-3 induced the production of specific anti-JJ3 antibodies.

To test whether the antibodies can cross react, the IgGs induced by peptide JJ and peptide JJ-1 were assayed by ELISA using the JJ peptide coated on the ELISA plate. As shown in FIG. 2A and FIG. 2B, the IgGs induced by the JJ peptide (FIG. 2A) or the JJ-1 peptide (FIG. 2B) were found to bind the JJ peptide coated on the ELISA plate. These results indicate that the antibodies produced by JJ-1 peptides can cross-react with the JJ peptide, suggesting that antibodies induced by peptides of the invention will be able to recognize various subtypes of hemagglutinins.

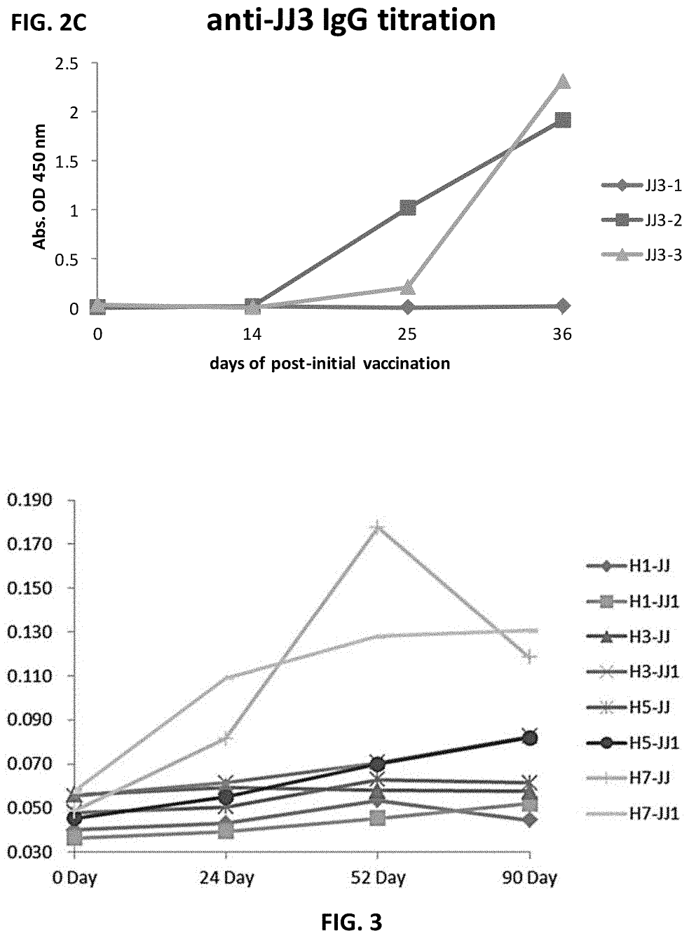

Indeed, the antibodies generated by peptide vaccines of the invention can bind various subtypes of hemagglutinins. As shown in FIG. 3, the antibodies generated with JJ peptide or JJ-1 peptide reacted with H1, H3, H5, and H7 subtypes of hemagglutinins. These results support the notion that antibodies generated with peptide vaccines of the invention will have a broad spectrum against various subtypes of hemagglutinins. Accordingly, peptide vaccines of the invention are universal vaccines against various influenza viruses.

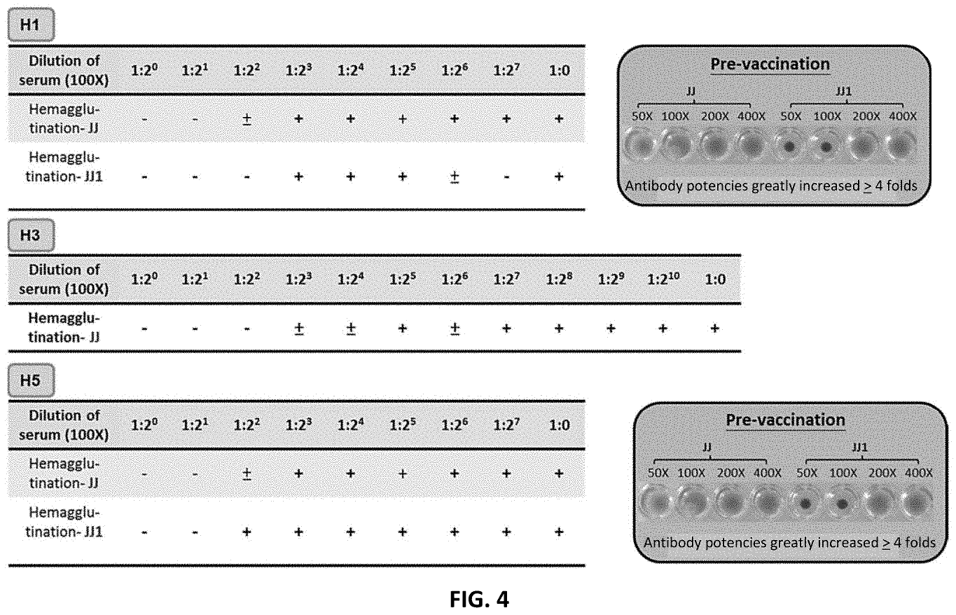

In addition to binding a broad spectrum of hemagglutinin subtypes, these antibodies were also found to be able to inhibit hemagglutination induced by influenza viruses. As shown in FIG. 4, these antibodies were able to inhibit hemagglutination induced by influenza viruses of the H1, H3, and H5 subtypes. In addition, the results show that the antibodies induced by JJ and JJ-1 are at least 4 times more effective in inhibiting hemagglutination than background of pre-immunization. These results validate the approaches of the invention and prove that peptides of the invention can be used to induce antibodies that can prevent influenza virus infection. The antibodies induced by peptides of the invention have a broad-spectrum against various hemagglutinins. Thus, peptide vaccines of the invention can induce broad-spectrum antibodies.

The fact that various variants of the JJ peptide all can induce antibodies that can react with a broad-spectrum of HA subtypes indicate that certain amino acid residues (e.g., F-3 and F-9 in JJ) are not involved in the MHC class II binding. These residues (e.g., F-3 and F-9 in JJ) are also not involved in TCR and MHC class II-peptide complex interactions because substitutions at these residues did not compromise their abilities to induce antibodies. TABLE II shows the alignment of these peptides:

| Peptide | Sequence | SEQ ID NO |
|---|---|---|
| JJ | GLFGAIAGFIE | 2 |
| JJ-1 | GLLGAIAGPIEF | 3 |
| JJ-2 | GLMGAIAGPIEF | 4 |
| JJ-3 | GLLGAIAGPIEGGW | 5 |
| JJ-4 | GLHGAIAGLIENGW | 6 |
| Consensus | GLXGAIAGXIE | 7 |

Based on the sequence alignment (TABLE 2), one can arrive at a consensus sequence (GLXGAIAGXIE, SEQ ID NO:8, wherein X stands for any amino acid residue), which will be sufficient for inducing antibodies against a broad-spectrum of HA subtypes. That is, a peptide having this consensus sequence will be a good peptide vaccine for inducing antibodies against a broad spectrum of HA subtypes. While the consensus sequence probably represents a minimal sequence, one skilled in the art would appreciate that longer peptides containing this minimal sequence may also be used. For example, additional amino acid residues may be added to the N- and/or C-terminal of these peptides. Similarly, these peptides may be part of a fusion protein.

One skilled in the art would appreciate that peptides of the invention may be used as a vaccine, which may be administered orally or by other routes (e.g., injection). In addition, these peptides may be used together with other components (e.g., adjuvants or other formulation agents) to function as a vaccine.

Methods for various procedures are known in the art. The following specific examples illustrate exemplary embodiments. However, one skilled in the art would appreciate that these specific examples are for illustration only and that modifications or variations are possible without departing from the scope of the invention.

### In Silico Analysis of HA Epitopes

Several modeling approaches are available for analyzing peptide-MHC class II bindings. For example, to model the binding of potential epitopes from various subtypes of hemagglutinins to the MHC class II molecules from different alleles, one can use the tools available at IEDB “MHC-II Binding Predictions,” http://tools.immuneepitope.org/mhcii/.

### Peptide (Antigen) Production

Antigen peptides of the invention are short peptides, which contain the epitope with 11-15 residues. The actual peptides for use as vaccines can be 11-15 residues long, or with additional residues on the N-terminal and/or C-terminal. In addition, these peptides may be coupled to other moieties to enhance the bioavailability and/or immunogenicity. These peptides can be readily synthesized chemically. In addition, these peptides may be produced by expression from host cells. The procedures for such productions are routinely available in the art.

### Antibody Generation

In this example, the peptide antigens in accordance with embodiments of the invention were used to induce antibody formation in mice. BALB/c and C57BL/6 mice (6-8 weeks old) were obtained from BioLASCO Taiwan (Taipei, Taiwan) or National Laboratory Animal Center (Taipei, Taiwan). The mice were randomly divided into six groups (3 mice in each group): a negative control group EXPO (LLVEAAPLDDTT; SEQ ID NO:8), Exp1 (JJ-1), Exp2 (JJ-2), Exp3 (JJ-3), Exp4 (JJ-4), and Exp5 (JJ).

Each peptide (Exp0-Exp5) at a dose of 45 ug/100 ul was mixed (at 1:1 ratio in volume) with Freund's complete adjuvant to obtain six different antigen solutions. Each group of mice was injected with a different peptide (Exp0-Exp5) solution as an antigen. For the first immunization, each mouse was given a subcutaneous injection with 100 μL (containing 45 of peptide each) of the above prepared antigen solution.

The second injection was given 10-14 days after the first injection. The solution for the second injection was prepared by mixing each peptide (6 different peptides, Exp0-Exp5, each at 1:1 mix) in Freund's incomplete adjuvant. The injections were repeated for subsequent immunizations every 10-14 days after the previous injections until antibodies could be detected in sera, which took from 3-6 injections.

Mouse sera were collected at the following time points: preimmunization and at the middle time point between two adjacent injections. Once the antibodies became detectable in sera, the serum collections were performed every two weeks until antibodies could no longer be detected, which typically took about half a year. The serum collections were performed from the cheeks of the mice. Each time, 100 uL or less of blood was collected and the collection frequency was about once every two weeks. At the end of the experiments, mice were euthanized with carbon dioxide.

### Immunologic Ab Titration

Antibody titers were determined using ELISA. First, a capture antibody (e.g., JJ peptide or various subtypes of hemagglutinins or fragments thereof) was coated on a 96-well microplate (100 μL per well of the diluted capture antibody). The plate was sealed and incubated at 4° C. overnight. Then, 100 μL of sample (e.g., antiserum) or standards in sample dilution buffer were added per well. The plate was sealed and incubated at room temperature for 1 hr.

After incubation, 100 μL of a detection antibody (e.g., a horseradish peroxidase (HRP) coupled antibody), diluted in antibody dilution buffer, was added to each well. The plate was sealed and incubated at room temperature for 1 hr. Then, 200 μL of substrate solution (3,3′,5,5′-tetramethylbenzidine (TMB)) was added to each well. The reaction mixtures were incubated for 20 minutes at room temperature, without exposing the plate to direct light.

After the reaction, 50 μL of stop solution (acid solution) was added to each well. Then, the optical density of each well was measured immediately, using a microplate reader set to 450 nm.

### HI Assays

The Hemagglutination Inhibition (HI) assay is based on the ability of HA antigen (on the surface of the influenza virus) to agglutinate red blood cells (RBC), thereby preventing red blood cells from precipitating. Antibodies that specifically bind HA (e.g., at the sialic acid-binding region or the stem region of hemagglutinin) can prevent agglutination, thereby allowing precipitation. The assay may be performed in 96 well round bottom plates with freshly prepared RBC from an animal (e.g., guinea pig RBC).

For example, blood from guinea pigs is washed with PBS and collected by centrifugation at 800 rpm for 5 minutes. The washing is repeated 2 more times. The washed blood is then diluted with PBS to make a final working solution of 0.5-0.75% RBCs in PBS for the assays.

An HA antigen (e.g., H3 viral antigen) solution was prepared by diluting the HA antigen with PBS to make serial dilutions. Hemagglutination assay was performed by gently mixing the HA antigen solution with the RBCs solution in a round-bottom 96-well microplate. The reaction mixture was incubated at room temperature for 30-60 minutes. Then, the HA titers were measured. Results are scored by observations: agglutination results in cloudy wells (i.e., no RBC precipitation), while inhibition of agglutination permits RBCs to clot and precipitate, resulting in a “button” of red cells at the bottom of the well.

To assay anti-HA titers of a serum sample, 4-8 HA units of the above HA antigen solution was diluted with PBS in a round-bottom 96-well microplate. To the wells were added serially diluted anti-sera ranging from 2× to 128× dilutions. The reaction mixture was incubated at room temperature for 10-15 minutes. Then, 0.5-0.75% RBCs solution from guinea pigs were added, and the mixtures were cultured for 30-60 minutes. The anti-sera HI titers were measured. The HI titer of a serum sample is the reciprocal of the last dilution which prevents agglutination (i.e., forms a button RBC precipitate). For example, if a 64× dilution allows the formation of a button of RBC precipitate, but the 128× dilution does not, then the HI titer is 64.

Embodiments of the invention have one or more of the following advantages. Embodiments of the invention use short peptides that can induce antibodies against influenza viruses. The peptides can be made protease-resistant, thereby making it possible to use oral administration routes for these peptide vaccines. Even though a vaccine of the invention uses a short peptide, these short peptides are surprising antigens and can induce anti-viral antibodies.

While embodiments of the invention have been illustrated with limited number of examples, one skilled in the art would appreciate that these examples are for illustration only and that other modifications or variations are possible without departing from the scope of the invention. Therefore, the scope of protection should be limited by the attached claims.

1. An antigenic peptide or protein, comprising the sequence of GLXGAIAGXIE (SEQ ID NO:7), wherein X is independently any amino acid residue, and wherein the antigenic peptide or protein has an ability to induce an antibody against influenza virus.

2. The antigenic peptide or protein according to claim 1, wherein the influenza virus includes H1, H3, H5, and H7 subtypes.

3. The antigenic peptide or protein according to claim 1, wherein the antigenic peptide or protein comprises the sequence of JJ (SEQ ID NO:2), JJ-1 (SEQ ID NO:3), JJ-2 (SEQ ID NO:4), JJ-3 (SEQ ID NO:5), or JJ-4 (SEQ ID NO:6).

4. The antigenic peptide or protein according to claim 1, wherein the antigenic peptide or protein comprises the sequence of JJ-1 (SEQ ID NO:3), JJ-2 (SEQ ID NO:4), JJ-3 (SEQ ID NO:5), or JJ-4 (SEQ ID NO:6).

5. The antigenic peptide according to claim 1, wherein the antigenic peptide or protein is formulated as an oral vaccine or an injection vaccine.

6. A method for inducing an immunity against influenza virus, comprising administering a vaccine to a subject in need thereof, wherein the vaccine comprises the antigenic peptide or protein according to claim 1.

7. The method according to claim 6, wherein the influenza virus includes H1, H3, H5, and H 7 subtypes.

8. The method according to claim 6, wherein the administering is via oral administration.

9. The method according to claim 6, wherein the administering is via injection.

10. The antigenic peptide according to claim 2, wherein the antigenic peptide or protein is formulated as an oral vaccine or an injection vaccine.

11. The antigenic peptide according to claim 3, wherein the antigenic peptide or protein is formulated as an oral vaccine or an injection vaccine.

12. The antigenic peptide according to claim 4, wherein the antigenic peptide or protein is formulated as an oral vaccine or an injection vaccine.

13. A method for inducing an immunity against influenza virus, comprising administering a vaccine to a subject in need thereof, wherein the vaccine comprises the antigenic peptide or protein according to claim 2.

14. A method for inducing an immunity against influenza virus, comprising administering a vaccine to a subject in need thereof, wherein the vaccine comprises the antigenic peptide or protein according to claim 3.

15. A method for inducing an immunity against influenza virus, comprising administering a vaccine to a subject in need thereof, wherein the vaccine comprises the antigenic peptide or protein according to claim 4.

16. The method according to claim 13, wherein the influenza virus includes H1, H3, H5, and H 7 subtypes.

17. The method according to claim 14, wherein the influenza virus includes H1, H3, H5, and H 7 subtypes.

18. The method according to claim 15, wherein the influenza virus includes H1, H3, H5, and H 7 subtypes.

19. The method according to claim 13, wherein the administering is via oral administration.

20. The method according to claim 14, wherein the administering is via oral administration.
